(19) United States
(12) Patent Application Publication
(10) Pub. No.: US 2003/0167007 A1
(43) Pub. Date: Sep. 4, 2003

(54) Apparatus and method for spectroscopic examination of the colon

(76) Inventor: Amir Belson, Cupertino, CA (US)

Correspondence Address: Johney U. Han, Morrison & Foerster LLP, 755 Page Mill Road, Palo Alto, CA, 94304-1018, US

(21) Appl. No.: 10/327,259

(22) Filed: Dec. 20, 2002

Related U.S. Application Data
(60) Provisional application No. 60/347,695, filed on Jan. 9, 2002.

(51) Int. Cl. A61B 6/00

(52) U.S. Cl. 600/473; 600/476; 600/478

(57) ABSTRACT

Apparatus and methods for spectroscopic examination of the colon are described herein. A spectroscopy device comprising an illumination device and an image capture device is integrated directly into a steerable endoscope or colonoscope. Alternatively, the spectroscopy device and the steerable colonoscope can be separate instruments that are functionally combined for performing endoscopic spectroscopy. The steerable colonoscope uses serpentine motion to facilitate rapid and safe insertion of the colonoscope into the patient's colon, which allows the endoscopic spectroscopy method to be performed more quickly and more safely. The spectroscopy can be performed by autofluorescence, dye-enhanced fluorescence or any other known spectroscopy techniques. Other imaging technologies that use light with a wavelength outside of the visible range may also be used. The reflected light information can be used to create a three-dimensional mathematical model of the patient's colon and the location of any lesions identified during the initial examination.

### CROSS-REFERENCE TO RELATED APPLICATIONS

This application claims the benefits of priority to U.S. Provisional Patent Application Serial No. 60/347,695 filed Jan. 9, 2002, the entirety of which is incorporated herein by reference.

### FIELD OF THE INVENTION

The present invention relates generally to methods and apparatus for medical diagnosis. More particularly, it relates to methods and apparatus for medical diagnosis of diseases of the colon and other organs using spectroscopic examination.

### BACKGROUND OF THE INVENTION

Endoscopic spectroscopy is an emerging technology for diagnosis of cancer and other diseases within a patient's body. Spectroscopic examination can be used to identify lesions that are not readily visible using white light endoscopy and/or to diagnose or differentiate tissues of suspected lesions that are found using white light endoscopy or other techniques. Auto fluorescence is a spectroscopic technique that illuminates a patient's tissues with one or more excitation frequencies and measures and/or images the natural fluorescence of the tissues. Differences in the natural fluorescence can be used to distinguish between normal cells and certain types of diseased cells. Dye-enhanced fluorescence is a spectroscopic technique in which one or more special fluorescent marker dyes are applied to the tissues either topically or systemically. The tissues are then illuminated with one or more excitation frequencies and the fluorescence of the tissues is measured and/or imaged. Differences in the uptake of the fluorescent marker dyes can be used to identify lesions and/or to distinguish between normal cells and certain types of diseased cells. Other known spectroscopic techniques can also be used. The following U.S. patents, each of which is incorporated herein by reference in its entirety, describe various spectroscopic techniques that can also be used in connection with the present invention:

U.S. Pat. No. 5,421,337 Spectral diagnosis of diseased tissue

U.S. Pat. No. 6,129,667 Luminal diagnostics employing spectral analysis

U.S. Pat. No. 6,096,289 Intraoperative intravascular and endoscopic tumor and lesion detection biopsy and therapy

U.S. Pat. No. 6,174,291 Optical biopsy system and methods for tissue diagnosis

U.S. Pat. No. 6,129,683 Optical biopsy forceps

U.S. Pat. No. 6,066,102 Optical biopsy forceps system and method of diagnosing tissue

U.S. Pat. No. 5,762,613 Optical biopsy forceps

U.S. Pat. No. 5,601,087 System for diagnosing tissue with guidewire

U.S. Pat. No. 5,439,000 Method of diagnosing tissue with guidewire

U.S. Pat. No. 5,383,467 Guidewire catheter and apparatus for diagnostic imaging

U.S. Pat. No. 5,413,108 Method and apparatus for mapping a tissue sample for and distinguishing different regions thereof based on luminescence measurements of cancer-indicative native fluorophor

U.S. Pat. No. 5,827,190 Endoscope having an integrated CCD sensor

U.S. Pat. No. 5,769,792 Endoscopic imaging system for diseased tissue

U.S. Pat. No. 5,647,368 Imaging system for detecting diseased tissue using native fluorescence in the gastrointestinal and respiratory tract

U.S. Pat. No. 5,590,660 Apparatus and method for imaging diseased tissue using integrated auto fluorescence

U.S. Pat. No. 5,507,287 Endoscopic imaging system for diseased tissue

Systems have been developed which combine a spectroscopic examination device with an endoscope, such as a colonoscope. Some systems allow the spectroscopic images to be superimposed onto the images produced by standard white light endoscopy. While these endoscopic spectroscopy systems represent an important advance in the diagnosis of cancer and other diseases, current systems are subject to many of the same limitations as standard white light endoscopy. In particular, currently available colonoscopes suffer from difficulties in insertion of the colonoscope and difficulties in determining and documenting the position of the suspected lesions within the patient's colon. In addition, the physician uses the white light for vision, to guide the colonoscope, and then has to stop and perform the spectroscopic exam, thus it is time consuming.

U.S. Pat. No. 6,129,667 describes a system for luminal diagnostics employing spectral analysis for creating a tissue map of a body lumen within a patient, such as a blood vessel the colon, small intestines, stomach or esophagus. The system uses radio-frequency, magnetic resonance or ultrasonic tracking techniques for tracking the position of the spectrometer device as it passes through the lumen in order to construct a three-dimensional map of the tissue based on the reflectance and/or absorption of light at the lumen wall. While this system addresses to some degree the need for determining and documenting the position of suspected lesions detected within the patient's body lumen, the usefulness of this information would be somewhat limited in connection with mapping the tissues of the colon because the position is determined with respect to external reference points. It does not inform the operator where the device is relative to the colon. In addition, the colon is somewhat mobile within the patient's abdomen and it can move subject to peristalsis and other forces; consequently it would be more advantageous to map the tissues of the colon and the position of suspected lesions based on internal reference points and landmarks that are fixed relative to the colon even though the organ itself is subject to movement within the patient's body. In addition, this prior art system does not address the difficulties of inserting the colonoscope through the torturous path of the colon or of accurately navigating the colonoscope back to the point of the suspected lesion for further diagnostic studies or surgical intervention.

Commonly owned and copending U.S. patent application Ser. Nos. 09/790,204 filed Feb. 20, 2001(now U.S. Pat. No. 6,468,203); 09/969,927 filed Oct. 2, 2001; and 10/229,577 filed Aug. 27, 2002, each of which is incorporated herein by reference in its entirety, describe a steerable colonoscope with multiple articulating segments that are controlled to move with a serpentine motion that facilitates rapid and safe insertion and withdrawal of the colonoscope with a minimum of contact and stress applied to the colon walls. In addition, the control system of the steerable colonoscope has the ability to construct a three-dimensional mathematical model or map of the colon as it advances through lumen under control of the operator. The three-dimensional mathematical model of the colon and the location and nature of any lesions identified in the course of an initial colonoscopic examination can be stored and used for accurately navigating the colonoscope back to the point of the suspected lesion for further diagnostic studies or surgical intervention. The technology described therein can also be used in conjunction with the methods and apparatus of the present invention to facilitate examination and diagnosis of the colon wall by endoscopic spectroscopy. These patent applications, and all patents and patent applications referred to herein, are hereby incorporated by reference in their entirety.

### SUMMARY OF THE INVENTION

In keeping with the foregoing discussion, the present invention takes the form of methods and apparatus for performing a spectroscopic examination of a patient's colon and for creating a three-dimensional map of the colon wall and the location and nature of any suspected lesions that are found during the spectroscopic image analysis.

The spectroscopy aspect of the invention can be performed by autofluorescence, dye-enhanced fluorescence or any other known spectroscopy techniques. Other imaging technologies that use light with a wavelength outside of the visible range may also be used.

The spectroscopy device can be integrated directly into the steerable colonoscope. Alternatively, the spectroscopy device and the steerable colonoscope can be separate instruments that can be functionally combined for performing endoscopic spectroscopy, for example by inserting the spectroscopy device through the working channel of the steerable colonoscope or through a channel dedicated to the spectroscopy device.

In a preferred embodiment, the present invention utilizes the steerable colonoscope described in copending U.S. patent application Ser. Nos. 09/790,204 (U.S. Pat. No. 6,468,203); 09/969,927; and 10/229,577, which have been incorporated by reference. The steerable colonoscope described therein provides a number of additional benefits for performing endoscopic spectroscopy according to the present invention. The steerable colonoscope uses serpentine motion to facilitate rapid and safe insertion of the colonoscope into the patient's colon, which allows the endoscopic spectroscopy method to be performed more quickly and more safely. In addition, the steerable colonoscope has the capability to create a three-dimensional mathematical model of the patient's colon and the location of any lesions identified during the initial examination. This information can be used to quickly and accurately return the colonoscope to the location of the identified lesions for further diagnostic studies or surgical intervention.

The endoscopic spectroscopy methods and apparatus of the present invention can also be applied to any other endoscopy procedure including but not limited to: esophgoscopy, gastroscopy, duodenoscopy and bronchoscopy.

### BRIEF DESCRIPTION OF THE DRAWINGS

FIG. 1 shows a first embodiment of an endoscopic spectroscopy system according to the present invention that combines a fiberoptic spectroscopy device with a steerable colonoscope.

FIG. 2 shows a second embodiment of an endoscopic spectroscopy system with a spectroscopy device integrated directly into a steerable colonoscope.

FIG. 3 shows a schematic diagram of one embodiment for producing, transmitting, and receiving light through a single optical fiber.

FIG. 4 shows a schematic diagram of another embodiment for producing, transmitting, and receiving light through separate optical fibers.

### DETAILED DESCRIPTION OF THE INVENTION

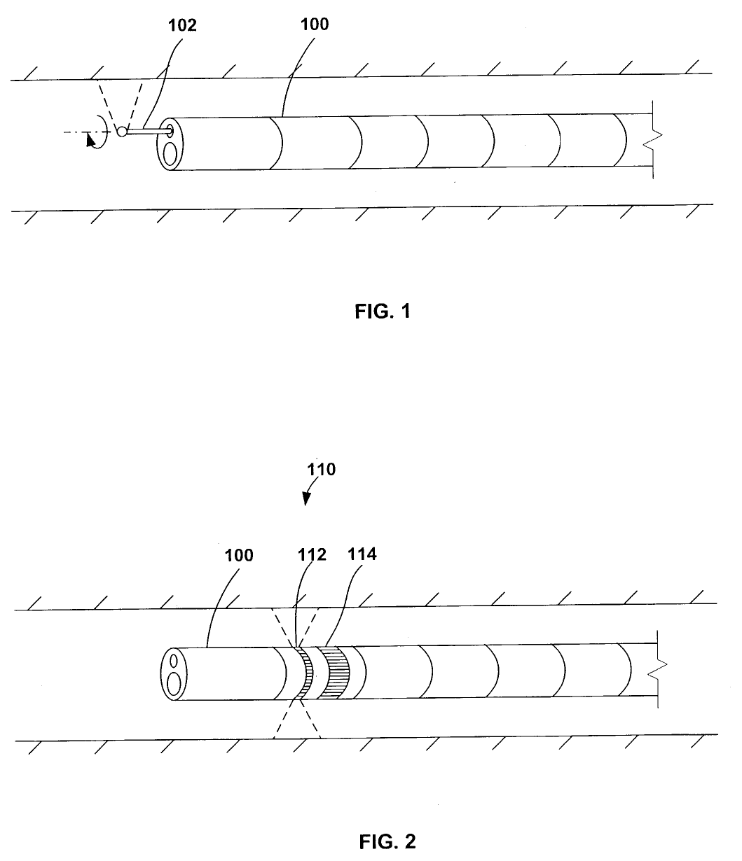

FIG. 1 shows a first embodiment of an endoscopic spectroscopy system according to the present invention that combines a fiberoptic spectroscopy device 102 with a steerable colonoscope 100. Preferably, the steerable colonoscope 100 is constructed as described in U.S. patent application Ser. Nos. 09/790,204 (U.S. Pat. No. 6,468,203); 09/969,927; and 10/229,577, with multiple articulating segments that are controlled to move with a serpentine motion that facilitates insertion and withdrawal of the colonoscope with a minimum of contact and stress applied to the colon walls. The steerable colonoscope 100 may be a fiberoptic endoscope or, more preferably, a videoendoscope that uses a CCD camera or the like to capture images of the inside of the colon. In addition, the control system of the steerable colonoscope 100 has the capability to construct a three-dimensional mathematical model of the colon as it advances through lumen under control of the operator. The three-dimensional mathematical model of the colon and the location and nature of any lesions identified in the course of an initial colonoscopic examination can be stored and used for accurately navigating the colonoscope 100 back to the point of the suspected lesion for further diagnostic studies or surgical intervention. The fiberoptic spectroscopy device 102 can be integrated directly into the steerable colonoscope 100 or the fiberoptic spectroscopy device 102 and the steerable colonoscope 100 can be separate instruments that are functionally combined for performing endoscopic spectroscopy, for example by inserting the fiberoptic spectroscopy device 102 through the working channel of the steerable colonoscope 100.

The fiberoptic spectroscopy device 102 delivers a beam of light with one or more excitation frequencies to illuminate the patient's tissues. The excitation frequencies may comprise UV, IR, NIR, blue light and/or other visible or invisible frequencies of light. The fiberoptic spectroscopy device 102 rotates to scan the tissues as the steerable colonoscope 100 advances or retreats. The fiberoptic spectroscopy device 102 captures the light that returns from the surface of the tissue by reflection, by natural fluorescence and/or by dye-enhanced fluorescence or other known spectroscopic technique. The steerable colonoscope 100 provides position information and the fiberoptic spectroscopy device 102 provides rotational information, as well as spectroscopic imaging data, to create a three-dimensional map of the spectroscopic properties of the tissues. The spectroscopic image of the colon captured by the fiberoptic spectroscopy device 102 may be superimposed on the white light endoscopic image of the colon captured by the steerable colonoscope 100 to facilitate analysis of the tissues and any suspected lesions identified. The spectroscopic examination and the white light endoscopic examination may be performed simultaneously if the wavelengths used for each are compatible and/or if the two images can be separated by appropriate optical or electronic filtering. Alternatively, the spectroscopic examination and the white light endoscopic examination may be performed intermittently or in an alternating fashion so that the wavelengths used do not interfere with one another. The three-dimensional map that is generated will enable the operator to return to an area that had some pathology or was suspected as having one in a previous exam, and then perform spectroscopic analysis of the area, and compare it to the previous picture from the same area.

FIG. 2 shows a second embodiment of an endoscopic spectroscopy system with a spectroscopy device 110 integrated directly into a steerable colonoscope 100. Preferably, the steerable colonoscope 100 is constructed as described in U.S. patent application Ser. Nos. 09/790,204 (U.S. Pat. No. 6,468,203); 09/969,927; and 10/229,577, with multiple articulating segments that are controlled to move with a serpentine motion that facilitates insertion and withdrawal of the colonoscope with a minimum of contact and stress applied to the colon walls. The steerable colonoscope 100 maybe a fiberoptic endoscope or, more preferably, a videoendoscope that uses a CCD camera or the like to capture images of the inside of the colon. In addition, the control system of the steerable colonoscope 100 has the capability to construct a three-dimensional mathematical model of the colon as it advances through lumen under control of the operator. The three-dimensional mathematical model of the colon and the location and nature of any lesions identified in the course of an initial colonoscopic examination can be stored and used for accurately navigating the colonoscope 100 back to the point of the suspected lesion for further diagnostic studies or surgical intervention.

Preferably, the spectroscopy device 110 is integrated directly into the steerable colonoscope 100, for example by integrating the spectroscopy device 110 into one of the articulating segments of the steerable colonoscope 100. In one particularly preferred embodiment, the spectroscopy device 110 extends around the circumference of the steerable colonoscope 100 and is capable of capturing spectroscopic data simultaneously from a 360-degree circle of tissue around the spectroscopy device 110. Alternatively, the spectroscopy device 110 can be configured to mechanically or electronically scan the tissues around the spectroscopy device 110 as the steerable colonoscope 100 advances or retreats.

The spectroscopy device 110 includes an illumination device 112 delivers a beam of light with one or more excitation frequencies to illuminate the patient's tissues. Preferably, the illumination device 112 delivers a ring of illumination in a 360-degree circle around the spectroscopy device 110. Preferably, the illumination device 112 includes one or more LED's or diode lasers or other known light source internal to the device to produce light at one or more excitation frequencies.

Alternatively, the illumination device 112 may use an external light source and a fiberoptic illumination cable to deliver the beam of light. The excitation frequencies may comprise UV, IR, NIR, blue light and/or other frequencies of light in a visible or invisible range. The spectroscopy device 110 includes an image capture device 114 to capture the light that returns from the surface of the tissue by reflection, by natural fluorescence and/or by dye-enhanced fluorescence or other known spectroscopic technique. Preferably, the image capture device 114 extends around the circumference of the steerable colonoscope 100 and is capable of capturing spectroscopic imaging data simultaneously from a 360-degree circle of tissue around the spectroscopy device 110. In a preferred embodiment, the image capture device 114 utilizes a CCD camera or the like internal to the device to capture the spectroscopic imaging data. The CCD camera may be configured to be sensitive only to the spectroscopic imaging frequencies of interest and/or appropriate optical or electronic filtering may be used. Alternatively, the image capture device may use a fiberoptic imaging cable and an external imaging device, such as a CCD camera, to capture the spectroscopic imaging data. The CCD camera may be configured to capture a wide-angle picture of the interior of the colon. Possible ways to capture a wide-angle picture include, but not limited to, using fish eye lens or spherical lens based camera.

The steerable colonoscope 100 provides position information and the spectroscopy device 110 provides spectroscopic imaging data to create a three-dimensional map of the spectroscopic properties of the tissues. The spectroscopic image of the colon captured by the spectroscopy device 110 may be superimposed on the white light endoscopic image of the colon captured by the steerable colonoscope 100 to facilitate analysis of the tissues and any suspected lesions identified. The spectroscopic examination and the white light endoscopic examination may be performed simultaneously if the wavelengths used for each are compatible and/or if the two images can be separated by appropriate optical or electronic filtering. Alternatively, the spectroscopic examination and the white light endoscopic examination may be performed intermittently or in an alternating fashion so that the wavelengths used do not interfere with one another. Another option is that the spectroscopic device will be located far enough from the tip so the light used for vision will not interfere with the spectroscopic exam.

The spectroscopic imaging data and the white light endoscopic imaging data may be viewed in real-time and/or recorded and stored for later analysis and diagnosis of any suspected lesions that are identified. In one preferred method of using the endoscopic spectroscopy system of the present invention, the spectroscopic examination takes place automatically as the steerable colonoscope 100 is advanced and retracted within the patient's colon. The operator is thus freed up to concentrate on manipulating the steerable colonoscope 100 to navigate the tortuous path of the colon and to perform the white light endoscopic examination. Both the spectroscopic imaging data and the white light endoscopic imaging data are recorded and stored together with the information of their exact location, for later analysis and diagnosis of any suspected lesions that are identified. The endoscopic spectroscopy system may also utilize pattern recognition software or the like to identify potential lesions from the spectroscopic imaging data and/or the white light endoscopic imaging data and to inform the operator that a particular portion of the colon warrants closer examination. This function will preferably be performed in real-time during the colonoscopic examination so that suspected lesions can be immediately investigated. In addition, this function may be performed on the recorded image data to enhance diagnostic accuracy.

In one preferred option the spectroscopic data that was recorded on the way in will be shown to the operator on the way out when the pictures shown are the pictures that were taken earlier from the location where the tip of the colonoscope is currently located. It will be achieved by using the three-dimensional mapping capability of the steerable colonoscope 100.

Another option is that the software that analyzes the spectroscopic data will identify suspected areas and when the colonoscope is withdrawn and arrives at the area of those suspected lesions (that were found on the way in), the system will signal to the operator about the suspected lesion and the operator will perform another spectroscopic exam or take a biopsy from the suspected area or lesion.

The stored imaging data from the endoscopic spectroscopy system and the three-dimensional mathematical model of the colon produced by the steerable colonoscope 100 can also be used for tracking progression of disease over time and/or for navigating the steerable colonoscope 100 to the identified lesions for subsequent surgical intervention

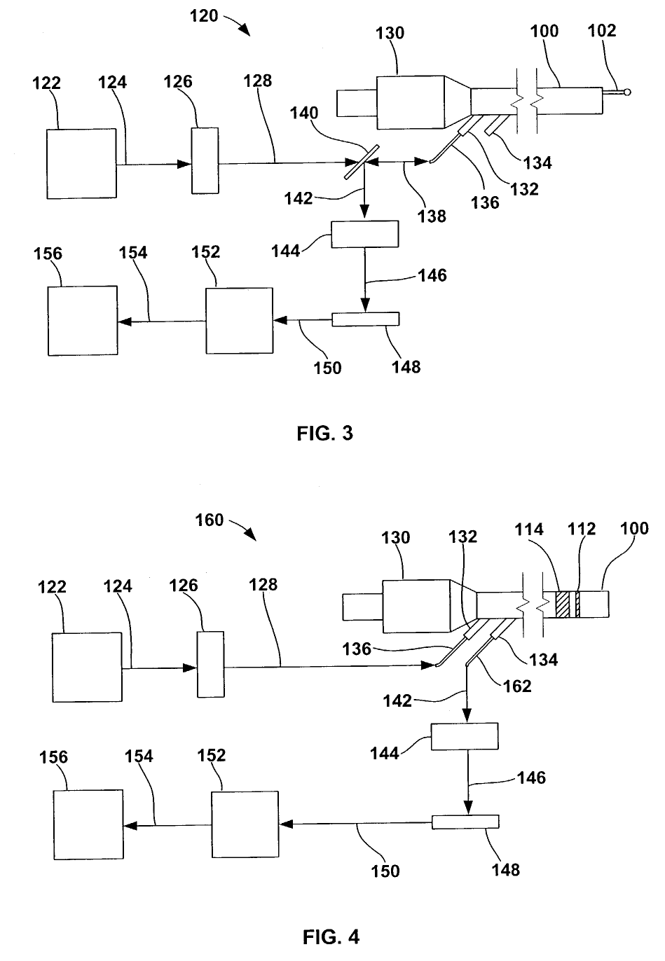

To produce, transmit, and receive the spectroscopic signals, a variety of assemblies may be used. FIG. 3 shows one embodiment in assembly 120 which may utilize a single fiberoptic cable, as shown in the embodiment of FIG. 1. A light source 122, which may include lasers, LEDs, etc., may be configured to produce a variety of different frequencies of light, e.g., UV, IR, NIR, blue light and/or other frequencies of light in a visible or invisible range, etc., depending upon the desired frequencies and types of signals to be generated. The light source 122 may generate light 124 which is transmitted through optical fibers which may then be passed through various filters and/or collimating lens assembly 126. This filtered and collimated light 128 may be passed through a beam splitter 140 and transmitted into the proximal end of the fiberoptic spectroscopy device 102. The fiberoptic cable 136 may optionally be routed into the colonoscope via an access port 132 or 134 located near or on the handle 130 of the colonoscope.

The distal end of the fiberoptic spectroscopy device 102 may be configured to be advanced or withdrawn relative to the colonoscope 100 itself. As described above, as the fiberoptic device 102 is rotated, it may emit the transmitted light or signal and also receive the reflected light with the spectroscopic information. This reflected light may be transmitted proximally back through optical fiber 136 and emitted as signal 138. This signal 138 may be reflected via the mirrored beam splitter 140 such that the reflected light 142 is directed towards filters and/or collimating lens assembly 144, which may be used to filter and/or collimate the signal. The filtered and reflected light 146 may then be directed towards a detector 148, e.g., a CCD detector, which may convert the light signals into electrical signals 150 which may be transmitted to a processor 152. The processed signal 154 may then be transmitted to a display unit 156 for relaying the reflected signals to the user.

Another embodiment for the transmission and processing of the spectroscopic information is shown in FIG. 4, which shows an assembly 160 similar to that of FIG. 3 but utilizing multiple fiberoptic cables, as shown for the embodiment of FIG. 2. In this variation, the light may be generated using the light source 122 and directed into the optical fiber 136. The light may be optically connected to the illumination device 112 near or at the distal end of the colonoscope 100. As described above, the illumination device 112 may be configured to direct the light radially about the colonoscope 100. The reflected signals may be incident upon the image capture device 114, which itself may be configured to be circumferentially positioned about the colonoscope 100. The image capture device 114 may be optically coupled to a distal end of a receiving fiberoptic cable 162. The signals may travel proximally through the cable 162 and be routed through the same access port 132 as optical fiber 136 or a second access port 134.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

I claim:

1. An endoscopic device for spectroscopically examining a hollow body organ, comprising:
an elongated body having a plurality of articulatable segments and a steerable distal portion, wherein each of the segments are configurable to assume a selected shape along an arbitrary path when the elongated body is advanced distally or proximally; and
a spectroscopic assembly having an illumination device and an image capture device adapted to receive an incident light reflected from a wall of the hollow body organ, wherein the spectroscopic assembly is positioned near or at a distal portion of the elongated body.

2. The endoscopic device of claim 1 wherein the illumination device comprises a light source disposed within the elongated body.

3. The endoscopic device of claim 2 wherein the light source comprises LEDs or laser diodes.

4. The endoscopic device of claim 1 wherein the illumination device comprises at least one optical fiber disposed within the elongated body, a proximal end of the optical fiber being in optical communication with a light source.

5. The endoscopic device of claim 4 wherein the light source comprises LEDs or laser diodes.

6. The endoscopic device of claim 4 wherein the light source is adapted to emit light having a frequency in a range selected from the group consisting of UV, IR, NIR, blue light, and visible light.

7. The endoscopic device of claim 4 wherein a distal end of the optical fiber is extendable beyond a distal end of the elongated body.

8. The endoscopic device of claim 7 wherein the optical fiber is adapted to rotate about a longitudinal axis of the optical fiber.

9. The endoscopic device of claim 1 wherein the image capture device comprises at least one optical fiber disposed within the elongated body.

10. The endoscopic device of claim 1 wherein the image capture device comprises a CCD camera.

11. The endoscopic device of claim 1 wherein the spectroscopic assembly is adapted to be advanced distally within a lumen defined within the elongated body.

12. The endoscopic device of claim 1 wherein the incident light comprises light emitted from the wall by a method selected from the group consisting of reflection, natural fluorescence, and dye-enhanced fluorescence.

13. A method of spectroscopically examining a hollow body organ, comprising:
positioning an elongated body having a plurality of articulatable segments and a steerable distal portion within the hollow body organ without impinging upon the hollow body organ;
illuminating an interior surface of the hollow body organ with an illumination device positioned upon a distal portion of the elongated body;
receiving a reflected light from the interior surface of the hollow body organ with an image capture device positioned upon the distal portion; and
processing the reflected light relayed by the image capture device.

14. The method of claim 13 wherein illuminating the interior surface comprises illuminating at least one LED or laser diode.

15. The method of claim 13 wherein illuminating the interior surface comprises illuminating a light having a frequency in a range selected from the group consisting of UV, IR, NIR, blue light, and visible light.

16. The method of claim 13 wherein illuminating the interior surface comprises extending a distal end of an optical fiber beyond a distal end of the elongated body.

17. The method of claim 16 further comprising rotating the optical fiber about a longitudinal axis of the optical fiber while illuminating the interior surface.

18. The method of claim 13 wherein receiving the reflected light comprises receiving the light with an optical fiber and transmitting the light to a proximal end of the fiber.

19. The method of claim 13 wherein receiving the reflected light comprises receiving light emitted from the interior surface by reflection or fluorescence.

20. The method of claim 13 further comprising applying a fluorescent marker dye to the hollow body organ prior to illuminating the interior surface of the hollow body organ.
